(19) United States
(12) Patent Application Publication
(10) Pub. No.: US 2020/0354409 A1
(43) Pub. Date: Nov. 12, 2020

(54) Adenoviral Polypeptide IX Increases Adenoviral Gene Therapy Vector Productivity and Infectivity

(71) Applicant: Kuopio Center for Gene and Cell Therapy Oy, Kuopio (FI)

(72) Inventors: Vesa TURKKI, Kuopio (FI); Saana LEPOLA, Kuopio (FI); Hanna LESCH, Kuopio (FI); Seppo YLA-HERTTUALA, Kuopio (FI)

(73) Assignee: Kuopio Center for Gene and Cell Therapy Oy, Kuopio (FI)

(21) Appl. No.: 16/569,742

(22) Filed: Sep. 13, 2019

Related U.S. Application Data
(63) Continuation of application No. 16/423,215, filed on May 28, 2019.
(60) Provisional application No. 62/844,175, filed on May 7, 2019.

(51) Int. Cl. C07K 14/005; C12N 15/86; A61K 48/00; C12N 5/073

(52) U.S. Cl. CPC C07K 14/005; C12N 15/86; A61K 48/0091; C12N 5/0603; A61K 48/0066; C12N 2710/00052; C12N 2510/00; C12N 2710/10023; C12N 2710/10034; C12N 2710/10022; C12N 2710/10043

(57) ABSTRACT

Producing adenovirus gene therapy vector in producer cells that express or over-express adenoviral polypeptide IX enables one to produce pIX-deleted adenovirus in suspension cell culture. Using producer cells that express or over-express adenoviral polypeptide IX also increases the yield of adenovirus vector, regardless of whether that adenovirus is pIX-deleted. Using producer cells that express or over-express adenoviral polypeptide IX also improves the resulting vector's transduction kinetics, reducing the number of pfu/target cell required to achieve a given level of transduction/infection, shortening the time the vector requires to transduce or infect a target cell, and shortening the time an infected target cell produces progeny virus.

### RELATED APPLICATIONS

This application claims priority from Saana LEPOLA et al., The Effert of Protein IX Over Expression to Stability and Infectivity of Adenoviral Vectors, United States provisional patent filing U.S. Ser. No. 62/844,175, filed 7 May 2019, the contents of which are here incorporated by reference.

### STATEMENT REGARDING FEDERALLY-SPONSORED RESEARCH OR DEVELOPMENT

None

### NAMES OF THE PARES TO A JOINT RESEARCH AGREEMENT

None

### SEQUENCE LISTING

This Specification incorporates by reference the electronic sequence listing files accompanying this application.

### STATEMENT REGARDING PRIOR DISCLOSURES BY THE INVENTORS

None.

### BACKGROUND

Adenoviridae family contains numerous viruses in several genera. They have a broad range of vertebrate hosts. Human adenoviruses are subdivided into seven species, and more than 50 distinct adenoviral serotypes have been described. Adenoviruses cause a wide range of illnesses, with most serotypes associated with the diseases of the respiratory system. Physically, adenoviruses are medium-sized (90-100 nm), non-enveloped viruses with an icosahedral nucleocapsid conformation. Their genetic material consist of a ˜36 kilobase (kb) double stranded DNA genome.

Adenoviruses enter into their host cells through endosomes. The virion has a unique spike or fiber associated with each penton base of the capsid that aids in virus attachment to a host cell via a receptor on the surface of the host cell.

Adenoviruses have long been a popular viral vector for gene therapy due to their ability to affect both replicating and non-replicating cells, accommodate large transgenes up to 8.5 kb. Since adenoviruses don't integrate their genetic material into the host cell genome, the transgene expression is transient. More specifically, they are used as a vehicle to administer targeted therapy in the form of recombinant DNA, RNA or protein, for example to treat malignant gliomas or bladder cancers.

The icosahedral capsid of adenovirus is composed of virus-encoded proteins. The capsid structure can be described as complex, but it is also well studied. The adenovirus capsid consists of 252 small building blocks called capsomers. The major coat protein of adenoviruses is the hexon protein and consequently the majority of the capsomers (240) are hexon capsomers. The remaining 12 penton capsomers are located at the fivefold vertices of the capsid. Hexon coat proteins form homo-trimers, which constitute the hexon capsomer. The hexons trimers are organized so that 12 trimers lie on each of the 20 facets of the capsid. A penton complex, formed by the peripentonal pentons and the base penton (holding in place a fiber), is at each of the 12 vertices.

Protein IX is a small multifunctional protein expressed by the members of the of Mastadenovirus family. In wild-type adenovirus, the central 9 hexons in a facet include 12 copies of Protein IX (pIX). Protein IX is not essential for viral replication. Thus, the art teaches to delete it from gene therapy vectors in order to increase the transgene capacity or reduce the likelihood of replication competent adenovirus (RCA) formation. See KOVESDI (2010); PARKS (2003). For example, PARKS (2004) notes, “In gene therapy studies, removal of pIX from the Ad vector backbone was used to increase the cloning capacity of E1-deleted Ad vectors.” See PARKS (2004) at Abstract. PARKS (2014) also notes, “Early studies suggested that Ad capsids devoid of pIX could not package full-length viral DNA,” yet “in contrast to previous reports, pIX deficient capsids can accommodate genome-sized DNAs.” See PARKS (2014) pg. 22 col. 2 (emphasis mine); see also SARGENT (2004). Similarly, nadofaragene radenovec, an adenoviral gene therapy vector carrying an interferon transgene, has a pIX⁻ genome (i.e., a genome from which pIX has been deleted to make room for the transgene and/or reduce the Replication-Competent Adenovirus risk).

Similarly, bacteriophage lambda deletion mutants are known to be more thermo-stable than wild-type phage. See COLBY (1981). The art thus teaches an adenovirus deletion mutant (d1313) which lacks the 5′ portion of the polypeptide IX gene. Id. Colby made this deletion mutant to increase viral stability, but surprisingly found that deleting the 5′ portion of the polypeptide IX gene makes the resulting virus substantially less thermo-stable than wild-type adenovirus. Id.; cf. RUSSEL (2009); ROSA-CALTRAVA (2001); ROSA-CALATRAVA (2003).

Specific modifications on adenovirus fiber proteins have been used to target adenovirus to certain cell types. MEULENBROEK (2004) uses pIX to affix green fluorescent protein onto the surface of virions, enabling one to track virus in vivo. Meulenbroek speculates pIX might enable one to also glue a monoclonal antibody or a cytotoxic onto adenovirus, making a targeted therapeutic. ROELVINK (2004) teaches to make a chimeric pIX which includes the native pIX base (which adheres to capsid) and non-native distal polypeptides which ostensibly target the virus to particular cell types. SALISCH (2017) teaches to make a malaria vaccine by attaching malaria-parasite antigen onto adenovirus surface using pIX as the molecular glue.

### BRIEF SUMMARY

The art teaches to manufacture adenoviral vector by deleting the E1 protein coding areas from the viral genome to make room for a therapeutic transgene, and then producing the resulting gene therapy vector in human HEK293 cells, which contain these E1 protein coding areas in their genome. Therefore these E1-deleted adenoviruses can grow in ti/m in HEK293 cells but not in vivo in patient cells. The art teaches also to delete the pIX coding region from the viral genome in order to increase the vector transgene capacity. Thus, the genome of some commercially-available adenoviral gene therapy vectors (e.g., ADSTILADRIN@ brand nadofaragene radenovec) do not contain the pIX coding region.

We have been developing recombinant adenoviruses (focusing with particular energy on serotype 5, or “Ad5”) manufacturing processes over the years. Traditional small scale processes to produce Ad use adherent HEK293 cells and cell culture flasks/bottles. These are useful for academic research, but are not easily scalable into commercial manufacturing. Our aim has been to develop a scalable manufacturing process for adenovirus vectors, for example serotype 5 adenoviruses (Ad5).

In the course of our process development work, we stumbled on a series of remarkable findings. Perhaps most significantly, we found that producing adenovirus gene therapy vector in producer cells that express or over-express adenoviral polypeptide IX enables one to produce pIX-deleted adenovirus in suspension cell culture at a surprisingly high yield. We also found that using producer cells that express or over-express adenoviral polypeptide IX increases the yield of adenovirus vector, regardless of whether that adenovirus genome is pIX-deleted. We also found that using producer cells that express or over-express adenoviral polypeptide IX improves the resulting adenoviral vector's transduction kinetics: the adenovirus needs fewer pfu/target cell to achieve a given level of transduction/infection, the adenovirus transduces or infects target cells more quickly, and infected target cells produce progeny virus more quickly. Our findings thus provide a way to fundamentally improve adenoviral gene therapy vector manufacturing.

Our invention thus pertains to, among other things, increasing the productivity, infection kinetics and infectivity of adenovirus (and particularly, adenoviral vector) by expressing pIX in the producer cells.

### BRIEF DESCRIPTION OF THE FIGURES

FIG. 1 compares the number of mass spectrophotometer spectra exhibited by HEK293 cells transduced with a pix-deleted adenovirus (i.e., an adenovirus with a genome from which pix has been deleted). Abbreviations: SC=Spectral Counting, the number of MS2 spectra associated with Protein IX (“pIX”). Ad A: pix-deleted adenovirus. Ad A 2: Infection with pix-deleted adenovirus in serum-free condition. Ad B: Control adenoviral vector, with a genome which contains pix. Statistical: vs Ad A 2 vs Ad B: pval_Ade=1.392955e-24. cell vs media: pval_comp=1.119278e-08. rep1 vs 2 vs 3: pval_rep=0.962930.

FIG. 2 compares the infectivity of each of two adenoviral gene therapy vectors (one with the pIX coding region and one without) in broad MOI range (vg/cell), each vector produced either in normal HEK293 cells or in HEK293-pIX(TF) producer cells. x axis=MOI; y axis=% of target cells infected or transduced.

FIG. 3 compares the infectivity of each of two adenoviral gene therapy vectors (we here call them “Ad A” and “Ad B”) produced in normal producer cells, and in producer cells transfected with a pIX-coding plasmid to transiently express pIX.

FIG. 4 shows flow cytometry result from infected cells stained with anti adenovirus antibody. It shows a cell population which appears at the later phases of complete infection. It thus compares time to lysis for target cells transformed with the various adenoviral gene therapy vectors of FIG. 3.

FIG. 5 is a schematic of a plasmid used to express pIX.

FIG. 6 is a color photograph of stained transfected producer cells.

FIG. 7 shows a PAGE separation of purified (CsCl+dialysis) adenovirus stocks stained with an anti-pX monoclonal antibody. Track 1: size markers. Track 2: Ad A (adenovirus lacking a pIX coding region) produced in pIX-expressing HEK293 producer cells. Track 3: Ad A produced in normal (pX-negative) HEK293 producer cells. Track 4: Ad B (adenovirus having a pIX coding region) produced in pIX-expressing HEK293 producer cells. Track 5: Ad B produced in normal (pX-negative) HEK293 producer cells.

FIGS. 8 and 9 shows photographs of various types of HeLa cell cultures, five days after infection/transduction with various types of adenovirus which were produced in various types of HEK293 producer cells. ARM=Adenovirus reference material. +pIX=Virus was produced in a HEK293 producer cell which expressed pIX. HeLa+pIX=Virus was administered to a HeLa target cell which expressed pIX. +pcDNA3.1=Virus was administered to a HeLa target cell transfected with an “empty” pcDNA3.1 plasmid, i.e., the plasmid lacking a pIX transgene.

FIG. 10 compares yield from adherent and suspension cultures using producer cells which do, and do not, express pIX.

The patent or application file contains at least one drawing executed in color. Copies of this patent or patent application publication with color drawing(s) will be provided by the Office upon request and payment of the necessary fee.

### DETAILED DESCRIPTION

The art teaches to manufacture adenoviral gene therapy vector by deleting the E1a and E1b protein coding areas from the wild-type adenoviral genome to make room for a therapeutic transgene. The art similarly teaches to delete the pIX coding region from the viral genome in order to increase the vector transgene capacity. Thus, for example, ADSTILADRIN@ brand nadofaragene radenovec, a commercially-available adenoviral gene therapy vector, has a genome which does not contain the pIX coding region.

### Example 1—HEK293 Cells Provide Complementation

The HEK293 cell line was established in 1973 by transforming human embryonic kidney (“HEK”) cells with sheared adenovirus type 5 DNA. A 4.5 kb piece of adenoviral DNA integrated into chromosome 19 of the HEK genome, creating the HEK293 cell line. The 4.5 kB piece of adenoviral DNA in the HEK293 genome contains the adenoviral genes e1a, e1b and ix. It represents about 11% of the far 5′ end of the adenovirus serotype 5 genome.

HEK293 cells include the adenoviral genes e1a, e1b and ix. Therefore, E1-deleted adenoviruses can grow in HEK293 cells but nor in normal human cells (which do not have adenoviral genes integrated into the chromosomal DNA). E1-deleted adenoviruses thus reduce the risk of forming infective (replication-competent) virus. The art refers to E1-deleted adenoviruses as “conditionally replicative,” meaning the virus is able to replicate only conditionally, i.e., in a host cell that provides the required complementation functions missing from the viral genome, and not able to replicate in cells which do not provide the required complementation functions. Deleting the adenoviral genes e1a, e1b and ix from the viral genome also increases the size of the transgene the vector can properly package.

We transduced HEK293 cells with either of two different adenoviral gene therapy vectors, “Ad A” or “Ad B”. Ad A has an adenoviral genome from which pix was deleted. Ad B has an adenoviral genome with an intact, expressed pix gene.

After three days, we separated the media from the transduced cells. We lysed the cells and loaded them onto a gel; cell-free culture media was loaded as such.

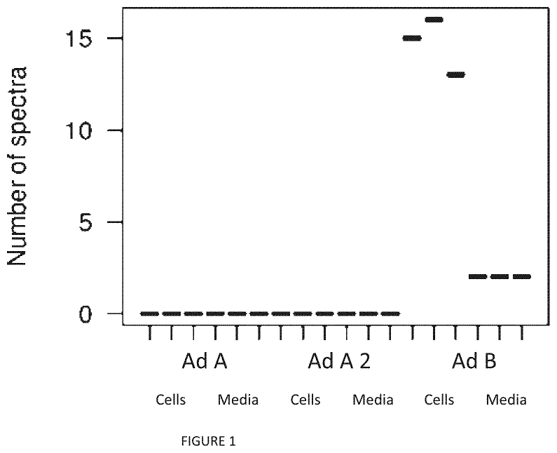

FIG. 1 compares pIX levels produced by HEK293 cells transduced with Ad A in serum-containing media (lane “Ad A”), Ad A in serum-free media (lane “Ad A 2”) or Ad B.

Our data show that with serum, Ad A does not lead to detectable pIX expression. These data also show that despite carrying the adenoviral pix gene, HEK293 cells do not express Protein IX. Thus, adenoviral vectors which are early-region deleted, and which are produced in HEK293 cells, do not have pIX in their capsids. Our mass spectrometry studies confirm that Protein IX is not observable in HEK293 cells, nor in adenoviral vectors which are pix-negative (i.e., have a genome from which pix has been deleted) which are produced in HEK293 cells. We thus found that despite the fact that HEK293 cells contain a pix coding sequence, HEK293 cells do not in fact express Protein IX and there is no detectable Protein IX. After a literature search, we found that this observation has been reported in the literature also.

Removing serum (momentarily, in order to synchronize cell cycles) from the culture medium does not change this. See FIG. 1 at column Ad A 2.

Using a virus which includes an intact, expressible pix gene provides measurable Protein IX. See FIG. 1 at column Ad B.

We performed another mass spectrometry study (data not shown), which showed that purified Ad A virions carry no detectable pIX, but wild-type adenovirus does.

### Example 2—Suspension Culture

We have done extensive process development work using single-use bioreactor systems. Over the course of five years, we made at least forty six (46) batches of adenovirus in single-use CultiBagRM™ bioreactors. The process included culturing of mammalian cell lines in roller bottles or shaker flasks, transfer of cells into a single-use bioreactor, expansion of the suspension-adapted cells in the bioreactor and infection to that the cells producerecombinant adenovirus. Virus material has been harvested by releasing intracellular viruses from the cells by chemical lysis followed by digestion of the host cell DNA with endonucleases. Resulting virus can be then subjected to downstream purification process. We have produced several recombinant adenoviruses, including adenovirus vectors where various parts of the early region of the genome have been deleted. On average, our HEK293 cells in suspension culture have produced about 3.16×10⁴±2.61×10³ viral particles/cell.

### Example 3—Suspension vs Adherent Culture

We compared the productivity of suspension and adherent cell culture systems for manufacturing vector. To do this, we used a serotype 5 adenovirus. As with Example 1 above, we used a early-region deleted adenovirus, i.e., the viral genome was modified to delete the E1a, E1b and pIX regions at the 5′ end of the wild-type adenovirus genome, as described by Ahmed et al (2001). Our adenovirus thus had an E1a-, E1b- and pix-negative genome. The vector was constructed using standard DNA manipulation techniques, and the viral genome also incorporates also some adenovirus serotype 2 (“Ad2”) genetic sequences.

We compared manufacture of this vector in various suspension culture systems, using 1-5 L working volumes and several small-scale, MOI-varying tests in shaker flasks. Surprisingly—and frustratingly—we found that yield and productivity were markedly low in each of these batches. The maximum productivity was 6×10³ vp/cell. This was an order of magnitude below our historical average (see Example 1) of 3.16×10⁴ vp/cell.

We replaced suspension culture with adherent culture. We found this achieved remarkably higher vector production in small scale, using adherent HEK293 cells in T-flasks using DMEM with 10% FBS. Vector production was up to two orders of magnitude higher using adherent culture rather than suspension culture.

We achieved the highest productivity (9.7×10⁴ vp/cell) using adherent culture conditions. See Table, Suspension/Adherent Process Comparisons. Our results show adherent culture was up to two orders of magnitude more productive than suspension culture. We did further suspension studies (data not shown), but none of those remarkably improved the markedly-low productivity of suspension culture compared to adherent culture.

Suspension / Adherent Process Comparisons

| Cell | | | |
|---|---|---|---|
| Culture Type | Volume/Type | Vp/Cell | Comment |
| Adherent | 175 cm² flask | 9.7 × 10⁴ | DMEM + 10% FBS, MOI |
| | | | 400, 48 h (Testing MOI |
| | | | 40-400, DOI 32-76 h) |
| Suspension | 21. Perfusion | 3 × 10⁴ | Schering-Plough, |
| | Stirred Tank | | MOI 40, DOI 80 h. |
| | | | CD293 + 1.1 mM Ca+ |
| | 30 ml, Shaker | 4 × 10³ | Ex-Cell medium |
| | Flask | | (Testing MOI, DOI, Ca+) |
| | 30 ml, Shaker | 1.1 × 10⁴ | CD293 medium w/o Ca+, |
| | Flask | | MOI 40, 48 h (Testing MOI |
| | | | 40-400, DOI 32-72 h) |
| | 30 ml, Shaker | 1.6 × 10⁴ | CD293 + 1.1 mM Ca+, |
| | Flask | | MOI 40, 48 h (Testing |
| | | | MOI, DOI, Ca+ conc.) |
| | 21, CellReady | 2 × 10² | CD293 + Ca + during |
| | Perfusion ST | | the infection, MOI 40, |
| | Bioreactor | | 48 h. Broken cells. |
| | | | Perfusion by ATF. |
| | 5L Perfusion | 5.5 × 10³ | EX-Cell medium, |
| | Cultibag/1L | | MOI 100, 48 h |
| | Wave | | |
| | Bioreactor | | |
| | 5L Perfusion | 6 × 10³ | Cell Growth in Ex-Cell, |
| | Wave | | CD293 + Ca+ during infection. |
| | Bioreactor | | MOI 40, DOI 48 h |

DOI = duration of Infection.
All experiments done with HEK293 cells.

The reason for low productivity in suspension was not known.

### Example 4—pIX Improves Infectivity

We used different vector genome doses to transduce target HEK293 cells. The numbers of transduced cells were counted 48 hours post-transduction (see table pIX increases Infectivity). Our data show that infectivity per viral genome increases in vectors which are produced in producer cells which express pIX.

We then made a similar experiment (see Table, Vector Made in pIX-Expressing Producer Cells Is More Infective in example 5), again comparing the infectivity of each of two adenoviral gene therapy vectors, Ad A (pix-deleted) and Ad B (pix-containing), each virus produced either in a normal (pix-negative) producer cell or a producer cell transfected with a plasmid expressing pIX. In contrast to our earlier experiment (comparing a range of MOIs), in this test we used a single MOI only, but tested a greater number of replicates to achieve greater statistical reliability of the results.

pIX increases Infectivity
Infectivity of Vector When Used to Infect at Varying vg/cell

| | |
|---|---|
| I | II |

Ad B (control adenovirus viith pIX)

| | | |
|---|---|---|
| a | 10.72 | 1.1 |
| b | 53.6 | 5.0 |
| c | 214 | 20.8 |
| d | 536 | 35.4 |
| e | 1072 | 58.2 |

Ad B + pIX (control adenovirus produced in cell expressing pIX)

| | | |
|---|---|---|
| f | 6 | 3.6 |
| g | 30.4 | 12.9 |
| h | 121.6 | 49.7 |
| i | 304 | 55.2 |
| j | 608 | 71.1 |
| Ad A (test virus, lacking pIX) | | |
| k | 2.9 | 0.5 |
| 1 | 14.5 | 2.1 |
| m | 58 | 9.0 |
| n | 145 | 17.9 |
| o | 290 | 25.8 |

Ad A + pIX (test virus produced in cell expressing pIX)

| | | |
|---|---|---|
| p | 2.82 | 0.7 |
| q | 14.1 | 4.3 |
| r | 56.4 | 12.8 |
| s | 141 | 25.3 |
| t | 282 | 28.8 |

I = viral genomes/cell used to infect the d rget cells (vg/cell)
II = % of target cells infected (Mean) and positive for adenovirus

These data show that pix-deleted adenovirus genomes are more infective if produced in a producer cell which expresses pix. For example, compare the Table, lines k and p. Produced in a normal cell, Ad A, when used at infectionat 2.9 vg/cell infects only 0.5% of target cells. (line k) Produced in a pix-expressing cell, infection with 2.8 vg/cell infects 0.7% of target cells. (line p) That is, fewer viral genomes infect 40% more target cells, if produced in a pix-expressing cell.

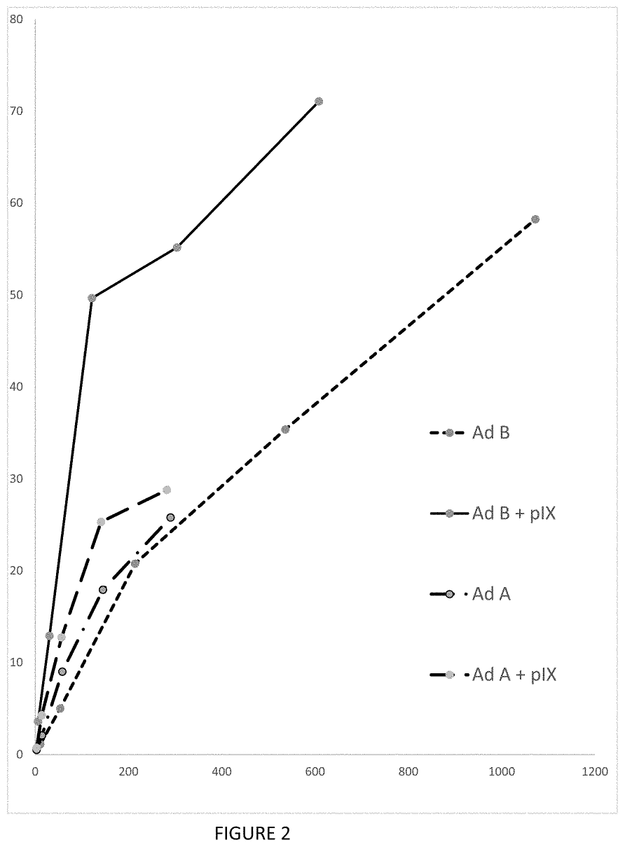

Similarly, compare Table, lines l and q. Produced in a normal cell, Ad A used to infect at 14.5 vg/cell infects only 2.1% of target cells. (line l) Produced in a pix-expressing cell, 14.1 vg/cell infection infects 4.3% of target cells. (line q) That is, slightly fewer viral genomes infect more target cells, if produced in a pix-expressing cell. Results are also shown in FIG. 2.

We have different hypotheses on the effect of pIX, which are not mutually exclusive. Without intending to be bound by theory we posit that:

1. When virus has been produced in pix over-expressing cells, and it is used for another round of infection, it has more pIX payload to release into a target cell after its entry. This pIX takes down host cell defenses, thus allowing more viruses to complete their life cycle than without pIX. Also when a virus is used to infect pix expressing producer cells, it is likely that not all producer cells are infected on the first round and antiviral mechanisms slow down/prevent the second round infection at least in some cells. The pIX helps by blocking the antiviral signals released by neighboring infected cells, thus keeping the producer cells open for the next infection round.

2. Producer cells that express Protein IX are better at properly packaging viral genome to make functional, infective virus. We believe that producer cells enable this by producing a greater-than-stoichiometric amount of Protein IX, i.e., more than 12 Protein IX molecules per viral genome. We posit that surplus Protein IX ensures that viral genomes are packaged efficiently and properly, increasing the relative yield of infectious particles per genome.

3. It is possible that adenoviruses lacking pix, particularly the Ad A used here, may be unable to enter into the host cell nucleus. Expression of pIX in the producer cells helps the viruses to establish productive infection by removing the intracellular blockage.

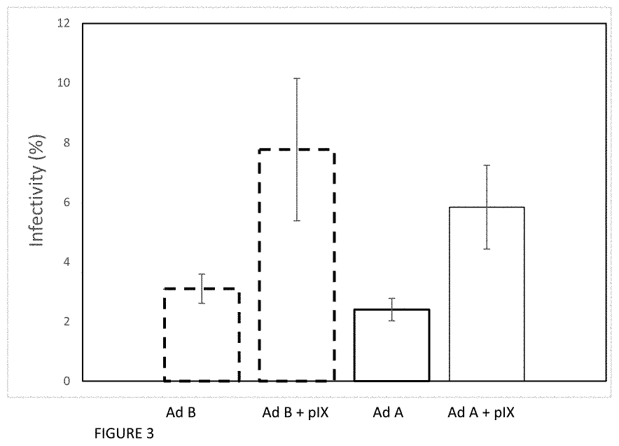

Our results for this repeat experiment are provided in FIG. 3 These data confirm that adenoviral gene therapy vector is perhaps 250% more infective if it is produced in producer cells which express the pIX polypeptide.

### Example 5—pIX Affects Infection Kinetics

In addition to researching how to make viral vector in greater volume, we have also been researching how to improve the resulting vector. To this end, we decided to study how the addition of pIX into our pix-deleted vector might affect virus stability.

We transfected HEK293 cells with plasmid containing an expressible pIX gene, creating HEK293-pIX cells which express pIX. We made HEK293-pIX cells that express pIX stably (“HEK293-pIX(stbl)”) and HEK293-pIX cells that express pIX transiently (“HEK293-pIX(TF)”).

We obtained an adenovirus gene therapy vector lacking a functional pix gene (here, “Ad A”), and an adenovirus gene therapy vector having a functional pix gene (here, “Ad B”), and manufactured each vector and a wild-type adenovirus (with a functional pix gene) in each of HEK293 cells and in HEK293-pIXcells.

We obtained HEK293 cells which, according to literature and our studies, do nor express pIX. We then transfected HEK293 cells with plasmid containing an expressible pIX gene, creating HEK293-pIX cells which express high levels of pIX either transiently (“HEK293-pIX(TF)” or stably (“HEK293-pIX(stbl)”). We also obtained an adenovirus gene therapy vector genome lacking a functional pIX gene (here, “Ad vector A”), and an adenovirus gene therapy vector genome having a functional pIX gene (here, “Ad vector B”), and wild-type adenovirus type 5 and manufactured each vector and the virus in both HEK293 cells and in HEK293-pIX cells.

We first describe our Materials and Methods, and then summarize our Results.

Materials And Methods

Materials

In this work, we used HEK-293 cells (Human embryonic kidney cells), available from American Type Culture Collection, catalog No. CRL-1573. These HEK293 cells contain the coding sequences for, but do not express, pIX. See e.g., GRAHAM (1977) pp. 65-66; SPECTOR (1980). HEK293 cells were used as a starting material to generate the stably pIX-expressing HEK293-pIX(stbl) line.

The pIX insert used in our work was created by amplifying it with polymerase chain reaction from the aforementioned HEK293 cells genome.

We used two adenovirus type 5 viral vectors. One vector (the “B” vector) contained the adenovirus vector genome with a complete pIX coding region. The second vector (the “A” vector) contained the adenovirus vector genome from which the pIX-encoding region had been deleted and which contained parts of Ad2 sequence. In addition to these, a wild-type (pIX containing) adenovirus type 5 was used.

Methods

### Plasmid Preparation Overview

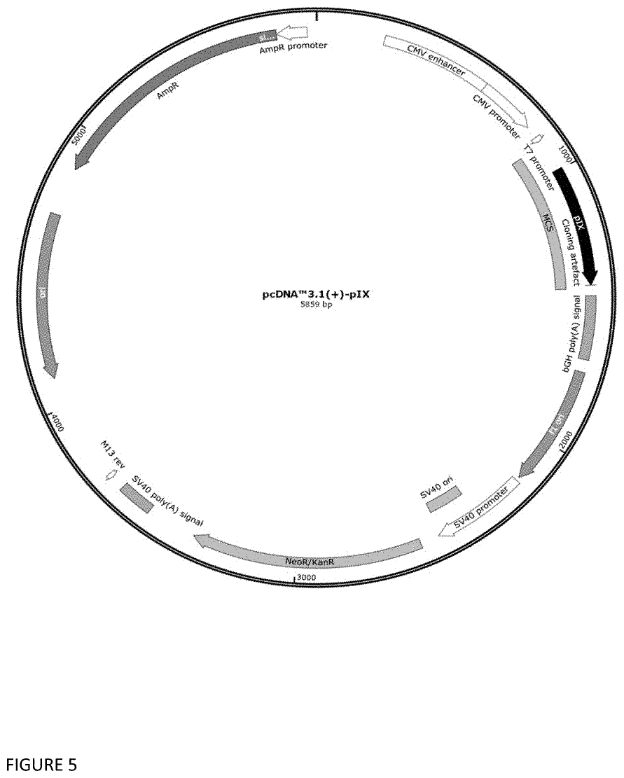

A transgenic plasmid containing the adenovirus protein IX (pIX) sequence was prepared. The pIX sequence was amplified from HEK293 cells genome by polymerase chain reaction and cloned into the pcDNA3.1™ vector base (commercially available from Adgene division of Thermo Scientific). The pIX transgene was inserted into XbaI+EcoRV opened pcDNA3.1 plasmid (see FIG. 5). pIX is under the CMV promoter, and its orientation is so that the coding area starts downstream of the CMV as shown in FIG. 5. pIX expression in cells was confirmed by staining the pIX with anti-pIX antibody after the cells had been transfected with the pIX-coding plasmid. In addition to this pIX positive signal, the intracellular location of pIX, in nuclei, also fits to what has been seen in case of high pIX expression in literature (speckled distribution of pIX in infected cell nuclei, Rosa-Calatrava et al., 2001).

### Digestion and Purification of the Protein Encoding Sequence IX

After amplification by PCR, the pIX DNA coding region was digested with the XbaI endonuclease. The digestion was done using 50 μl of PCR product suspended in CutSmart™ Buffer (New England Biolabs, Massachusetts, USA), using 60 Units XbaI (New England Biolabs) and nuclease-free, Molecular Biology grade Water (ThermoScientific, Massachusetts, USA). Incubation and inactivation was performed according to Table, Enzymes Used In The Preparation Of Plasmid pcDNA3.1-pIX (below).

After inactivation of the restriction enzyme, the sample was run on a 1% agarose gel (TopVision™ Agarose, Thermo Scientific) using SYBR Safe™ DNA gel stain (Invitrogen, California, USA) and 5 μl of Generuler™ DNA ladder mix (Thermo Scientific) as a size marker. The gel was run using a Horizon 11.1™ (Life Technologies, California, USA) at 110 V for 50 minutes. The gel was photographed using a ChemiDoc™ Touch Imaging System (Biorad, California, USA). Bands containing DNA were excised and DNA isolated using a Qiaquick™ gel extraction kit (Qiagen GmbH, Hilden Germany). Concentrations were measured with NanoDrop™ ND-1000 Spectrophotometer (Thermo Fisher Scientific).

Envzymes Used In The Preparation Of Plasmid pcDNA3.1-pIX
Incubation and Inactiva don Temperatures and Times

| | Reaction/Inactivation Condition | |
|---|---|---|
| Enzyme | Temperature (C) | Time (minutes) |
| XbaI | +37/+65 | 120/20 |
| PNK | +37/− | 30/− |
| EcoRV-HF | +37/− | 210/− |
| SAP | +37/− | 50/− |
| ligase | +22/+65 | 15/20 |
| SmaI | +25/+65 | 25/20 |

The DNA sample was then subjected to polynucleotide 5′-hydroxyl kinase treatment (PNK) to add a gamma phosphate to the 5′end of the insert. The reaction mixture consisted of a sample (56 μl) of buffer (T4 DNA ligase buffer+10 mM ATP, New England Biolabs), 10 Units of PNK (14 Polynucleotide Kinase 3′ phosphatase, BioLabs, Massachusetts, USA) and water. The reaction was incubated according to the Table, Enzymes Used In The Preparation Of Plasmid pcDN A3.1-pfX.

Digestion and Purification of prDNA 3.1™

A restriction enzyme reaction was performed to digest the plasmid template (7 μg pcDNA3.1™), in CutSmart™ buffer with 50 Units of XbaI and 50 Units of EcoRV-HF restriction endonuclease (New England Biolabs) and water. The reaction mixture was incubated according to Table. Enzymes Used In The Preparation Of Plasmid pcDNA3.1-pIX. After the incubation, the mixture was diluted with 40 μl of water, the above buffer and 5 Units of Shrimp Alkaline Phosphatase (SAP) to remove phosphates from the DNA chain ends, thereby preventing self-ligation. To the sample (70 μl), 14 μl of loading color was added. The sample was then pipetted into two wells on a 1% agarose gel. In addition, 6 μl of marker was pipetted onto the gel. The gel was run at 100V for 55 minutes. The digested plasmid DNA was isolated from the gel according to the instructions of the QIAquick™ gel extraction kit. Concentrations were measured with a NanoDrop™ spectrophotometer.

Ligation of Protein IX Sequence to pcDNA3.1

The ligation reaction consisted of pcDNA3.1™ plasmid (50 ng gel-purified plasmid), buffer (T4 DNA ligase buffer, containing 10 Mm of ATP), ligase (400 Units of T4 DNA Ligase, New England Biolabs), insert (41.6 ng gel-purified insert) and water. Incubation and inactivation conditions were according to Table, Enzymes used in the preparation of plasmid pcDNA3.1-pIX.

Transformation of Bacteria Using the pcDNA3.1-pIX Plasmid

The ligation sample was transformed into One Shot™ Cmnimax™ brand chemically-competent E. coli (Invitrogen) using the heat-shock method. Cells were thawed on ice, following which 2 μl of ligation sample was combined with 40 μl of cells. One μl pf Puc19 DNA plasmid (Invitrogen) was used as a positive control. Samples were allowed to stay on ice for 30 minutes. They were then heated at +42° C. for 30 seconds. The samples were then kept for 2 minutes. Then, 250 μl of SOC medium (Invitrogen) was added, and the tubes were incubated at 37° C. and 225 rpm for 70 minutes. Cells (100 μl) were plated on ampicillin plates, 50 μg/ml AMP (Sigma Chemical Co., Missouri, USA) and incubated at +37° C. for 16 h.

Colony-PCR for Screening the Colonies for Correct pcDNA3.1-pIX Clones

Samples from bacterial colonies were harvested from the plate in 50 μl culture medium (lysogeny broth (+AMP), Sigma-Aldrich, Missouri, USA) into the wells on a 96-well plate. The plate was incubated at +37° C. at 225 rpm for 2 hours and 45 minutes. The cultured colonies were subjected to PCR reactions according to the Table, Reaction Mixture Used In Colony PCR. The primers used are shown in the Table, Primers.

Reaction Mixture Used In Colony PCR

| | |
|---|---|
| F-518 5X Phusion ™ buffer (Finnzymes Oy) | 5 μl |
| Primer (from Table, Primers) | 10 pMol |
| dNTIPS (Thermo Scientific) | 4 nMol |
| DNA polymerase (Thermo Scientific) | 0.4 Units |
| Water | 2 μl |
| Total volume | 20 μl |

Primers

| | Sequence (5′ - 3′ | |
|---|---|---|
| Primer | direction) | Usage |
| F | GCCATGAGCACCAACTCG | Primers used in |
| | TTTGATGG | colony PCR reactions |
| R | TAGAAGGCACAGTCGAGG | Primers used in |
| | | colony PCR reactions |
| A (F) | GCCATGAGCACCAACTCG | Primers used |
| | TTTGATGG | for sequencing |
| B (R) | TAGAAGGCACAGTCGAGG | Primers used |
| | | for sequencing |
| C (F) | TAATACGACTCACTATAG | Primers used |
| | GG | for sequencing |
| F | CATGACCTTATGGGACTT | Primers used in |
| | TCCT | ddPCR for CMV |
| | | containing vectors |
| R | CTATCCACGCCCATTGAT | Primers used in |
| | GTA | ddPCR for CMV |
| | | containing vectors |
| Probe | /56 FAM/TCGCTATTA/ | Primers used in |
| | ZEN/CCATGGTGATGCGGT/ | ddPCR for CMV |
| | 3IABkFQ | containing vectors |

Abbreviations:
FAM : 6-carboxyfluorescein a/k/a 6-FAM
ZEN : the ZEN ™ brand quencher, commercially available from Integrated DNA Technologies Inc., Coralville IA USA
3iABkFQ : the 3′ Iowa Black ® FQ quencher, commercially available from Integrated DNA Technologies Inc., Coralville IA USA

The PCR was run with the program according to the Table, Program Used In Colony PCR, on a Peltier PTC-200™ thermal cycler (Bio-Rad).

Program used in colony PCR

| | Temp (C.) | Time (min:sec) |
|---|---|---|
| Initial denaturation | +98 | 3:00 |
| Denaturation | +98 | 0:10 |
| Hybridization | +62 | 0:20 |
| Extension | +72 | 0:18 |
| Thermal cycling (30 cycles) | as above | as above |
| Final elongation | +72 | 7:00 |
| Preservation | +4 | ∞ |

PCR products were separated on a 1% agarose gel (10 μl of product/well+2 μl loading buffer) at 120 V for 40 minutes. Also, we included a marker as described above. We photographed the gel as described above. On the basis of the gel, we selected the bacterial colonies containing pcDNA3.1-pIX plasmid to be cultivated. We placed the selected colonies in 4 ml of LB-AMP culture medium and we grew the culture at +37° C. at 170 rpm for 16 hours.

### Miniprep Purifications

In order to multiply the bacteria suspected of carrying the correct plasmid, we performed miniprep DNA purifications for the pcDNA3.1-pIX-transfected bacteria grown in 4 ml LB-Amp after the colony PCR. We used a miniprep kit from Macherey-Nagel GmbH, Germany. Sample concentrations were checked with a NanoDrop™ spectrophotometer.

Restriction Endonuclease Reactions to Confirm the pDNA3, 1-pIX Structure

The purified plasmids were subjected to SmaI restriction digestion to identify a plasmid prep with correct insert to use. The digestion reaction consisted of a plasmid (300 ng/reaction), 1×CutSmart™ (New England Biolabs), 10 Units of SmaI restriction endonuclease (New England Biolabs) and water. Incubation and inactivation conditions were according to Table, Enzymes used in the preparation of plasmid pcDNA3.1-pIX. Digested samples were separated on a 1% agarose gel as described above, using 20 μl sample and 4 μl of loading buffer per lane. In addition, 5 μl of marker was pipetted into one lane. The gel was run at 110 V for 45 minutes and 130 V for 15 minutes. The gel was photographed as described above. After the correct pcDNA3.1-pIX plasmid was confirmed by restriction enzyme digestion, it was sequenced (Gatc-biotech.com/lightrun). The primers used for this sequencing are shown in Table, Primers.

### Cell Culturing

HEK293 cells were used for both viral production and to assay the infectivity of the resulting virus. As cell culture medium, we used Dulbecco's Modified Eagle Medium (DMEM) supplemented with 10% fetal bovine serum (FBS), 2 mM glutamine and 1% penicillin/streptomycin (Gibco, N.Y., USA). Cells were grown at +37° C., 5% CO₂ in a Hera Cell 150™ incubator (Heraeus, Germany) and cultures were split twice per week. For cell counting, the culture medium was removed and the cells were washed with phosphate buffered saline (Gibco, N.Y., USA). The cells were dissociated using TrypLE Select™ (Gibco) and suspended in fresh culture medium. Cells were stained using trypan blue (Invitrogen) at a final concentration of 0.2%, and incubated at room temperature for 2 minutes. We counted cells using a Countess II™ cell counter (Invitrogen). We calculated the number of virus required for infection according to the number of cells obtained, using this equation:

$$virus \div [ mL media per well ] = [ viral genomes per cell \times cells per well ] \div viral genomes per mL media$$

Stably pIX-Expressing HEK293-pIX(Stbl) Cell Line

HEK293 cells were transfected using pcDNA3.1-pIX plasmid and cultured in the presence of a selection reagent (Geneticin, 200-600 μg/ml). A cell bank was manufactured and the expression of the pIX was confirmed on Western blot gels.

### Viral Productions

The purpose of virus production was to produce new batches of adenoviral vectors and adenoviruses, some of which would be produced in an intracellular environment characterized by the expression or over-expression of pIX. We made several separate manufacturing runs to verify that the differences in the vectors (if any) did not result from uncontrolled manufacturing variations.

### Adenovirus Vector and Virus Productions

Vectors and viruses were produced in adherent cell cultures using standard adenovirus production techniques with the exception of transfecting some of the cells before virus infections. We plated HEK293 and HEK293-pIX(stbls) cells onto 25, 75 or 175 cm² cell culture flasks or 500 cm², three-layer flasks (Thermo Fisher Scientific) at a density to provide about 70-90% confluence on the day of transfection. We plated a total of 1-5 flasks per virus. Some of the flasks were transfected with protein IX-expressing plasmid before virus infections.

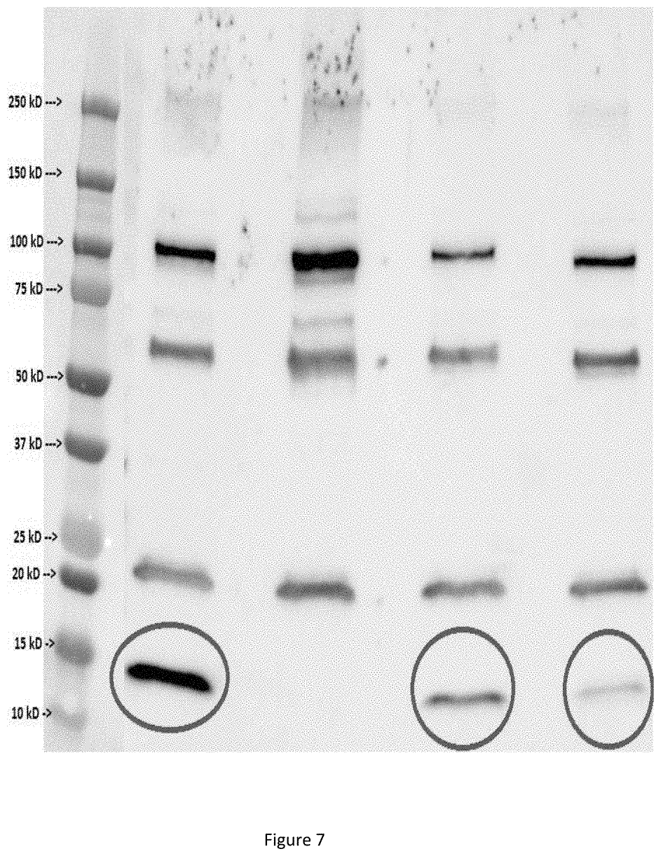

Anti-pIX antibody was used to confirm the presence or absence of pIX in purified (CsCl+dialysis) adenovirus stocks. FIG. 7 shows the results of these assays. Stains reveal the presence of pIX in adenovirus that includes a pIX-coding region, and adenovirus produced in producer cells expressing pIX, but not in adenovirus which both lacks a pIX coding region and is produced in a producer cell which does not express pIX. Our data confirm that Ad A, an adenovirus which does not code for pIX, does nor contain pIX unless the virus producer cells have been transfected with pcDNA3.1-pIX plasmid.

### Transfections

For the transfection, we replaced the cell culture medium into fresh medium on the day of transfection. The medium volume after the media change was about 50% of the standard medium volume recommended for the flasks in question. The plasmid bearing the pIX coding areas (100-200 ng/cm² culture area) was suspended in fresh media or NaCl solution (approx 3 ml per flask). We diluted of PEIpro or JetPEI (Polyplus™) polyethylenimine transfection reagent in equal volume. PEI was used in 1-2× mass ratio to plasmid. GFP or mCherry—containing plasmids were used as transfection controls for fluorescence microscopy confirmation of successful transfection. We added the diluted PEI into the diluted DNA, mixed and incubated the solution for 15-25 minutes. We then added the transfection mixture on to the cells. After 4 hours, we exchanged the media for fresh media containing 10% FBS.

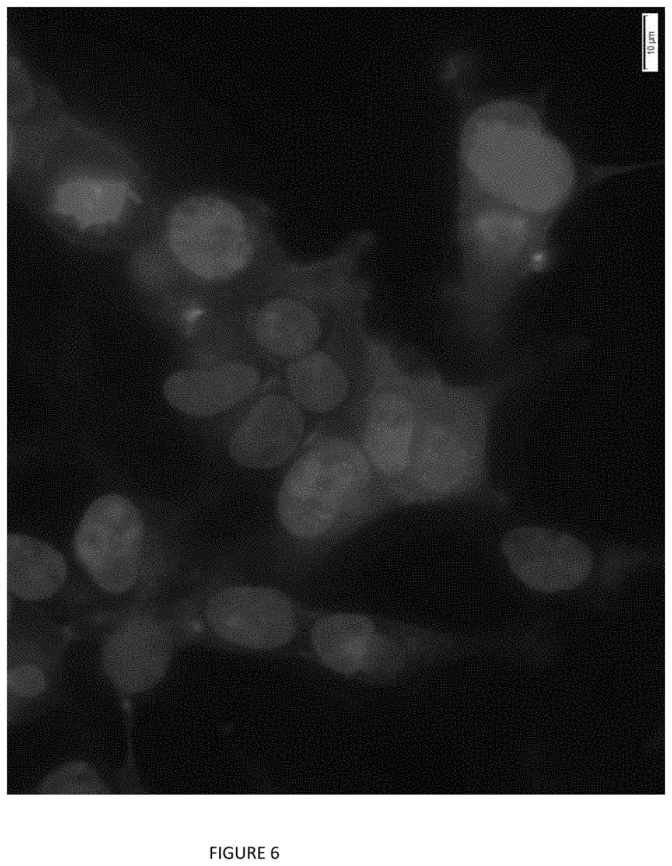

In order to confirm that transfection with pcDNA3.1-pIX plasmid leads to pIX expression, HEK293 cells were transfected and stained with anti-pIX antibody after 48 hours incubation. FIG. 6 shows our typical results. In addition to anti-pIX (secondary stained red), we also stained nuclei (blue) and cell tubulin (green). We studied the cells using a standard fluorescent microscope. FIG. 6 shows that the antibody recognizes proteins, and shows nuclear localization in similar manner as has been reported for pIX

### Vector and Virus Infections

To some of the transfected flasks, we added Ad vector B (having a pIX coding region). To other flasks, we added Ad vector A (lacking the pIX coding region), or wild-type adenovirus. Each was added at 40-200 virus particles or virus genomes/cell. We retained some of the flasks as controls (such as mCherry and GFP reporter flasks and random pcDNA3.1-pIX transfected flask). After 2 hours, we added culture medium to each flask up to the recommended culture volume. We then incubated the flasks for an additional 48-72 h. Infected cells were detached into culture medium and we centrifuged the medium at approximately 1100×g for 10 min at room temperature to pellet the cells. We re-suspended the pellets in 1-4 ml PBS, then lysed the cells and released the vectors/viruses by freezing at −80° C. and then to +20-37° C., repeated three times. We separated cell fragments by centrifugation (500-2000×g, 10-20 min, at +4° C.).

When needed, we then purified virus and vector particles from the supernatant. For viral purification, we made a CsCl gradient (6 ml of 1.45 g/ml CsCl and 14 ml of 1.33 g/ml CsCl) in an ultra filtration tube (Beckman, Calif., USA). We filled the tube with cell lysate supernatant and the CsCl gradient was ultra-centrifuged for 19 hours at 76,220×g, +21° C.) using an Optima™ LE-80K ultracentrifuge (Beckman Coulter) with an SW28™ rotor (Beckman) at 28,000 rpm. We used needle (such as Microlance™ 23 XG, Becton Dickinson, New Jersey, USA) and syringe (Terumo, Japan) to collect the virus band from the ultra-centrifuged tubes.

We then injected viruses into Slide-A-lyzer™ 10,000 MWCO dialysis cartridges (Thermo Scientific) and immersed the cartridges in 2 liters of sterile PBS. Dialysis buffer changes of different duration were performed for different batches from 4 hours to overnight in approx 2 liters volume to change the CsCl into PBS. We collected viruses from the dialysis cartridges on a needle, and then stored the viruses at −80° C.

We determined a titer for the virus material using a ddPCR titering assay. We examined transfection controls by fluorescence microscopy using an Olympus IX81, LUCPlan FLN 40×/0.60 Pl2∞/0-2/FN22 (Olympus Corp., Japan) to review transfection efficiency.

We also determined transfection efficiency by flow cytometry. For this, we washed the cells with PBS, dissociated the cells using TryPLE Select™ and re-suspended the cells in PBS. We then centrifuged the cells at 300×g for 5 minutes, and re-suspended pellets in 500 μl PBS. We then added 500 μl of 4% paraformaldehyde in PBS (Sigma-Aldrich) to the tubes and then incubated the tubes at +4° C. for 15 minutes. We pelleted the cells by centrifugation at 500×g for 5 minutes, followed by washing with PBS and centrifugation as above. The pellet was again re-suspended in PBS and we measured the amount of positive cells by flow cytometry.

Determination of Adenoviral Titer with ddPCR

The samples were subjected to DNase and Proteinase K treatments. The reaction mixture consisted of a sample (10 μl), DNAs (2U, Invitrogen) and buffer (DNAse buffer with 0.05 vol. % Pluronic F-68 (Gibco)). The mixture was incubated at +37° C. for 30 min after which it was inactivated at +95° C. for 10 min. Proteinase K was added (2U, Roche, Switzerland) and buffer. We then incubated at +50° C. for 30 min and then inactivated at +95° C. for 20 min. The reaction mixture for ddPCR is shown in the Table, Reaction Mixture Used In ddPCR, and the primers used are shown in the Table, Primers.

Reaction mixture used in ddPCR

| | |
|---|---|
| ddPCR Supermix ™ (Bio-Rad) | 11 μL |
| Primer (from Table, Primers) | 19.8 pMol |
| Probe (from Table, Primers) | 5.5 pMol |
| Sample | 5 μL |
| Water | q.s. |
| Total volume | 20 μL |

The reaction was run according to the manufacturer's instructions (Automated Droplet Generator, C1000 Touch Thermal Cycler, QX200 Droplet Reader, Bio-Rad). The program is shown in the Table, Program used in ddPCR above. The results were analyzed in the Quantasoft™ 1.7.4.0917 (Bio-Rad) program.

Proerain used in ddPCR

| | Temp (C.) | Time (min:sec) |
|---|---|---|
| Initial denaturation | +95 | 10:00 |
| Denaturation | +94 | 0:30 |
| Hybridization & Extension | +60 | 1:00 |
| Thermal cycling (39 cycles) | as above | as above |
| Final elongation | +98 | 10:00 |
| Preservation | +4 | oo |

### Western Blot and Coomassie Staining for Adenoviral Samples

The proteins contained in the viruses and/or cells were examined using both Western blot and/or Coomassie staining. In some cases samples were concentrated before analysis (Concentrator plus/vacufuge plus, Eppendorf, Germany) for 60 minutes at +60° C. We loaded samples with loading buffer (Laemmli, Bio-Rad) and heated them for 10 min at +96° C. We used Mini-PROTEAN™ TGX pre-cast gels, 4-20% (Bio-Rad), pipetting 22 of sample/well and additionally 8 of Precision Plus™ protein marker (Standard Dual Color, Bio-Rad). We ran the gels at 80 V for 15 min and then at 180 V for 30 min using a PowerPac™ Basic power supply (Bio-Rad) in sodium lauryl sulfate buffer (Bio-Rad). We then blotted gels onto Trans-Blot Turbo™ membrane, 0.2 m PVDF (Bio-Rad). We incubated the membranes for one hour in blotting solution (PBS with 5% milk powder (Valio, Finland) and 0.05% Tween™ 20 (Merck)). The blotting solution was changed to the primary antibody anti-pIX (α-pIX rabbit) serum (provided by Professors David Curiel and Igor Dmitriev, Washington University in St. Louis School of Medicine), diluted 1:500 in blotting solution. We then incubated the membranes at +4° C., 100 rpm for 20 hours. We washed the membranes with PBS (0.05% Tween™ 20 added) for 10 minutes, four times, followed by the addition of a secondary antibody (Goat anti-rabbit IgG (H+L)−HRP Conjugate, Bio-Rad) diluted 1:3000 the blotting. We then incubated the membranes at room temperature for 100 rpm for 3 hours. We then used a Chemi/UV/stain-free tray in a ChemiDoc™ Touch Imaging System (Bio-Rad) to digitize the images.

In Coomassie blue staining, we stained samples of gels of viruses of both yields and HEK-293 negative controls. We ran the gels as described above. After that, we fixed the gels in a mixture of ethanol and acetic acid (40% to 10%) for 15 min at 100 rpm. We then washed the gels four times, for 5 min each, with water and QC Colloidal Coomassie Stain (Bio-Rad) at +4° C. at 100 rpm for 20 hours. We then washed the gels four times for 10 min each with water, and then digitized them using the white tray of the ChemiDoc™ Touch Imaging System (Bio-Rad).

### Infectivity Test for Adenoviral Samples

HEK293 cells were pipetted onto a 12-well plate at 2.4×10⁵/well. To each well we added 1 ml of culture medium with 10% FBS. Plates were incubated at +37° C. at 5% CO₂ for about 24 hours. Cells were counted from one well/plate as previously described. Viruses were pipetted into wells at the desired amounts (40-200 vg/cell). In addition, as a negative control no virus was added. In addition to virus, we added serum-free growth medium to each well to produce a final volume of 500 μl. Plates were incubated at +37° C. in 5% CO₂. After two hours, we exchanged the media for 1 ml of fresh media with 10% FBS. We then incubated the cells for 46 hours at +37° C. in 5% CO₂.

The culture solution was aspirated and the cells were removed with 300 μl of TryPLE Select. We added 900 μl of PBS to the TryPLESelect and transferred the cells to Eppendorf tubes. To the mixture we added 2 mM MgCl₂ and 50 Units of benzonase (Merck Millipore, Denmark). The mixture was incubated at +37° C. for 10 min. Cells were centrifuged at 500×g for 5 minutes. To fix cells, we then added PBS and 500 μl of 1:1 acetone (VWR Chemicals, Pennsylvania, USA) and a mixture of methanol (Sigma-Aldrich). We allowed the cells to fix at +4° C. for 45 minutes. We then added 1 ml of PBS and stored the mixture at +4° C.

We then centrifuged the cells at 500×g for 5 minutes, washed with PBS and centrifuged again. We removed the supernatant and added 500 W of 1% BSA in PBS as blocking solution and centrifuged as above. We left 50 μl of the blocking solution in the tube and added 25 d of Adeno DFA Reagent™ anti-hexon monoclonal antibody (Millipore Corp, Massachusetts). We then incubated at +4° C. for 20 minutes, and then added 925 μl PBS and centrifuged as before. We removed the supernatant and re-suspended the pellet in 150 μl of PBS. We then pippetted samples onto a 96-well plate and read the using a CytoFlex S Ordiorflow cytometer (Beckman Coulter) and analyzed the results using CytExpert™ software.

### Cell Staining And Fluorescence Microscopy for pIX Expression Confirmation

HEK293 cells were plated on an Ibidi μ-Slide™#80826 8-well plate (ibiTreat GmbH, Germany) in 200 μl of DMEM 10% FBS). We allowed the slides to incubate overnight at +37° C. in 5% CO₂.

We made a transfection mixture of 0.28 μl of PEIpro in 100 μl culture medium supplemented with 0.2 μg of plasmid (pcDNA3.1-pIX) in 100 μl culture medium. The mixture was stirred and allowed to incubate at room temperature 20 min, after which it was added to the cells. After four hours, we exchanged the media for fresh media and then incubated the cells at +37° C. at 5% CO₂ for 48 hours. Cells were washed with PBS and fixed with 2% PFA. After 20 minutes, we added 0.1% Triton™ X-100 (Fluka, Switzerland) to the cells and incubated for 10 minutes at room temperature. The cells were then washed in blocking solution of 1% bovine serum albumin in PBS. We used an anti-pIX monoclonal antibody as the primary antibody, diluted 1:500 in blocking solution. We then incubated overnight at +4° C. The cells were washed twice and then treated with 1.98 mg/ml of Alexa Fluor 647 donkey anti-rabbit antibody, catalog #150075 (Abcam Limited, UK) and 1:500 diluted blocking solution, and allowed to incubate at room temperature for 1 hour. We then washed the cells twice with blocking solution.

We then added 150 μl of NucBlu™ (Invitrogen) to the wells, 2 drops/ml. We also added 1:250 DMLA, FITC-conjugated α-tubulin antibody blocking solution, catalog #Ab64503 (Abcam Inc.) and incubated for 1 hour. We then washed the cells with blocking solution and PBS. We photographed the samples using an Olympus™ IX81 fluorescence microscope with an oil lens (60×/1.35 UplanSApo ∞/0.17/FN26.5) and analyzed by CellSens™ standard software (Olympus).

### Results

While not the original aim of the study, we surprisingly found that over-expression of pIX in producer cells increased both the rate of vector production and the rate at which the resulting adenovirus vector transduces target cells. We also saw that these adenoviruses, when administered to target cells, show faster infection than their counterparts that were produced in normal (Protein IX-free) HEK293 cells.

We surprisingly and counter-intuitively also found that expression of pIX in producer cells has beneficial effects not only for producing pix-deleted virus, but also for the pix-containing virus, including producing wild-type adenovirus.

We surprisingly found that if producer cells have Protein IX, then the virus produced in those producer cells achieve a cytopathic effect (CPE, a sign of virus infection) faster than does virus produced in producer cells which lack Protein IX. More specifically, after a low multiplicity of infection (“MOI”), ARM virus showed CPE in pIX over expressing cells in 3 days, whereas normal HEK cells showed the CPE in four days.

We also found that if producer cells express pIX, then the producer cells produce vector much faster than do producer cells which do not express pIX.

We also surprisingly found that the vectors produced in pIX-expressing cells transduce target cells more efficiently than do similar vectors produced in producer cells which do not express pIX.

We conclude that an adenoviral gene therapy vector which includes pIX infects and transduces target cells more rapidly than a vector without pIX. To our surprise, we also saw an increase in the virus infectivity (infectivity/virus particle) of vectors produced in pIX expressing cells.

Our experiments evaluate manufacturing adenovirus gene therapy vector without pIX polypeptide, and also with pIX (either expressed as part of the adenovirus genome, e.g., as in the wild-type adenovirus genome, or on a discreet plasmid). Our results show that adenovirus vector which is manufactured in an environment with pIX polypeptide produces adenovirus viral vector particles that are more infective than those produced in an environment without pIX polypeptide. By increasing infectivity, we can reduce the number of vector particles needed to transform a required number of target cells. Increasing infectivity also reduces the lag time between administering a therapeutic dose of gene therapy vector and achieving a particular level of transgene expression.

Vector Made in pIX-Expressing Producer Cells is More Infective

Ad A (pix-deleted) and Ad B (pix-containing), two adenovirus gene therapy vectors, were each produced in either normal HEK293 cells (which do not express pX) or in HEK293 cells transfected to transiently express pIX. These four vectors were purified using CsCl gradient centrifugation and dialysis techniques. Vectors were titered using ddPCR method in order to find out the concentration of capsid-enclosed vector genomes. FIG. 3 illustrates the effect of pIX on infectivity. Our data show that producing a pix-deleted adenovirus in a pix-expressing producer cell more than doubles the infectivity of the resulting vector. Surprisingly, our data also show that producing a pix-containing adenovirus in a pix-expressing producer cell also more than doubles the infectivity of the resulting vector. This finding is surprising because the artisan would have expected that in a pix-expressing producer cell, the pix gene in the adenovirus genome would be redundant, providing no added benefit.

Vector Made in pIX-Expressing Producer Cells Is More Infective

| | Experiment # | | | |
|---|---|---|---|---|
| | (three independent | | | |
| | experiments, numbers | | | |
| | 1-3 refer to these in | | | |
| Virus | chronological order). | Inf | avg | s.d. |

Ad B

| | | | |
|---|---|---|---|
| Experiment 1 | 3.23 | 3.10 | 0.49 |
| Experiment 2 | 3.19 | | |
| Experiment 3 | 2.89 | | |

Ad B + pIX

| | | | |
|---|---|---|---|
| Experiment 1 | 10.40 | 7.77 | 2.39 |
| Experiment 2 | 6.11 | | |
| Experiment 3 | 6.80 | | |

Ad A

| | | | |
|---|---|---|---|
| Experiment 1 | 2.69 | 2.40 | 0.38 |
| Experiment 2 | 2.29 | | |
| Experiment 3 | 2.22 | | |

Ad A + pIX

| | | | |
|---|---|---|---|
| Experiment 1 | 6.46 | 5.84 | 1.41 |
| Experiment 2 | 4.30 | | |
| Experiment 3 | 6.76 | | |

Compiled Results

| | | | |
|---|---|---|---|
| | | | % INC |
| Ad B | 3.10 | 0.49 | — |
| Ad B + pIX | 7.77 | 2.39 | 250% |
| Ad A | 2.40 | 0.38 | — |
| Ad A + pIX | 5.84 | 1.41 | 240% |

Ad B = Adenovirus including the pIX coding region
Ad A = Adenovirus lacking the coding region
+ pIX = Producer cells which express pIX
% Inf = % of target cells infected
% INC = Percentage increase in infecdvity associated with vector produced in pIX-expressing host cells.
avg = Mean (average)
s.d. = standard deviation

### Example 6—pIX Speeds Target Cell Transduction

Expression of pIX in producer cells appears to produce viral vector which can more rapidly transduce target cells.

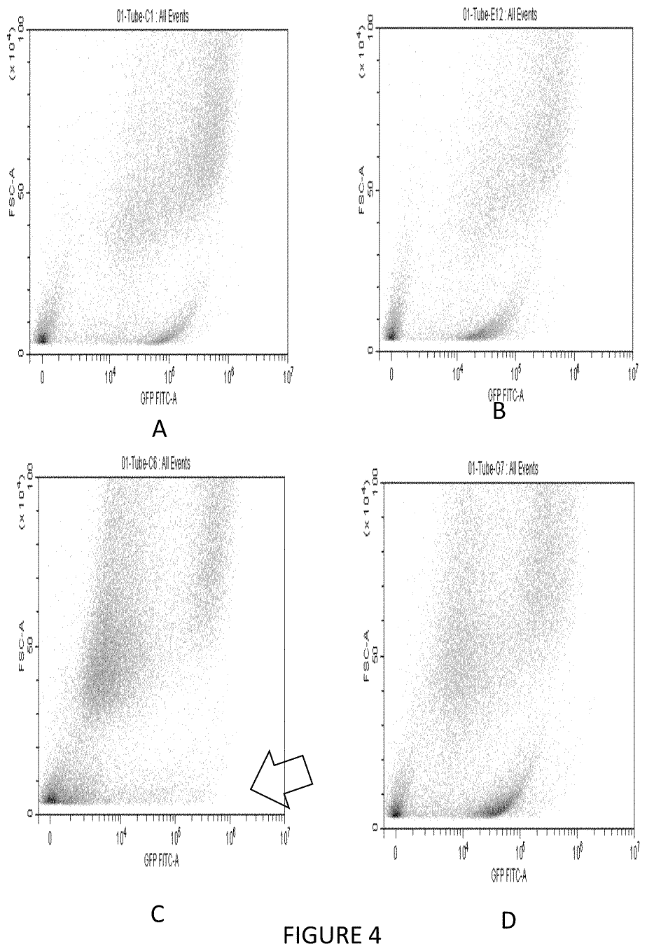

FIG. 4 shows flow cytometry analyses of target host cells transformed with each of four different adenoviral vectors. Panel A shows results for an adenoviral gene therapy vector which includes the pIX coding region in its genome (and thus expresses pIX polypeptide when produced). Panel B shows results for the same vector, produced in HEK293 cells transfected with a plasmid expressing the pIX polypeptide (and thus expresses the pIX polypeptide). Panel C shows results for adenoviral gene therapy vector made from a genome lacking pix, and produced in HEK293 cells, and thus lacking pIX when manufactured in HEK293 cells. Panel D shows results for the same vector, produced in HEK293 cells which were transfected with a plasmid expressing the pIX polypeptide; these virus particles thus have pIX when produced. For each scatter plot, the apparent end-point of viral production is shown by a dark cluster at the bottom of the scatter plot towards the middle of the x axis, indicating the population of lysing dying cells.

Each of the three vectors which include pIX when manufactured produce a plume of lysing or dying cells within the experimental timeframe. The one vector which completely lacked pIX (pIX-negative virus produced in a pIX-negative producer cell, Panel C) did not produce such a plume within the experimental time frame. The place where the plume would be expected to occur is indicated by the arrow in the figure.

These results show that adenoviral gene therapy vector is able to more rapidly transduce a population of target cells if the infecting gene therapy virions have pIX.

### Example 7—pIX Increases Infectivity

pIX can be used to increase virus infectivity several ways. pIX can be expressed or over-expressed in virus producer cells and the resulting virus produces an infection which seems to progress more efficiently or faster, to produce more infected cells in given time, as compared to virus produced in a pIX-free producer cell. On the other hand, viruses produced in cells which express pIX also seem to infect more cells when administered on target cells. In previous assays, we used a defined number of virus genomes per target cell. This raises the question of whether pIX really increases the infectivity per genome, or perhaps merely affects the genome titering efficacy through an unknown mechanism.

In order to obviate the genome titering phase, we produced pix-containing virus in an identical setting and equal volumes used previously (5 μl after 3× freeze-thaw and dilution to 1 ml). This virus was used to infect wells into which 7×10⁴ HEK293 cells had been seeded 2 days earlier. Infection times were 21-23 min. Approximately 2.8-4.8×10⁴ cells were analyzed from each well.

Our data shows that pIX increases the sheer number of infective units produced per volume in most cases. For wild-type Ad, the transiently transfected HEK293 led to increase in infectivity. For Ad B (adenovirus containing piv in its genome), the stably pIX-expressing cells increased transduction unit productivity the most.

We also observed a decrease in ARM infectivity in stably pIX expressing cells compared to standard (pIX-negative) HEK293 cells. This decrease was likely due to the observed sub-optimal culture density of the HEK293(stbl) used. Microscopy observations show that ARM replication speed in these cells was slightly increased compared to controls used in our earlier tests (data not shown). Ad A+pcDNA3.1 was an important control, showing that the expression plasmid alone (without pIX) was nor the reason for increased infectivity.

pIX Can Be Used mSeveral Ways To Increase Infectivity

| | | | | | |
|---|---|---|---|---|---|
| Virus | Target | % Inf. | W | s.d. | SD % |

Adenovirus Reference Material

| | | | | | |
|---|---|---|---|---|---|
| ARM | HEK293 | 6.95% | 1* | NA | NA |
| ARM + pIX | HEK293 | 16.11% | 2 | 0.0092 | 5.71% |
| ARM | HEK293- | 12.50% | 9 | 0.06885 | 55.10% |
| | pIX (TF) | | | | |
| ARM | HEK293- | 1.415%** | 2 | 0 00255 | 18.02% |
| | pIX (Stbl) | | | | |
| ARM | HEK293 | 6.95% | 1* | NA | NA |

Ad B (virus with pix gene)

| | | | | | |
|---|---|---|---|---|---|
| Ad B | HEK293 | 0.30% | 3 | 0.0017 | 56.10% |
| AdB + pIX | HEK293 | 1.51% | 9 | 0.0019 | 12.29% |
| AdB | HEK293- | 16.78% | 2 | 0.00920 | 5.48% |
| | pIX (stbl) | | | | |

Ad A (virus without pix in its genome)

| | | | | | |
|---|---|---|---|---|---|
| Ad A | HEK293 | 0.04% | 2 | 0.0004 | 100.00% |
| Ad A + pIX | HEK293 | 0.68% | 9 | 0.0004 | 5.19% |
| Ad A | HEK293- | 0.20% | 2 | 0.0008 | 40.00% |
| | pIX (TF) | | | | |
| Ad A + pcDNA3.1 | HEK293 | 0.01% | 1 | NA | NA |
| empty plasmid | | | | | |

Virus = Virus used “+pIX” indicates the virus was produced in a producer cell which expressed pIX.
Target = Type of target cells transduced (or infected) by virus.
% Inf = Percent of cells infected at 48 hours.
W = number of infected wells,
s.d. = Standard Deviation
SD % = Percentage variance in the Standard Deviation.
ARM = Adenovirus Reference material (wild-type adenovirus with pIX coding region)
* = The cell pellet from replicate well was lost during the staining.
** = HEK293-pIX(stbl) was observed to grow in suboptimal density at the dine of infection.

### Example 8—pIX does not Cause RCA

These data raised for us the question of whether the increase in vector productivity we observed could have been be due to pIX expression causing the formation of replication-competent adenovirus (“RCA”), e.g., a wild-type virus.

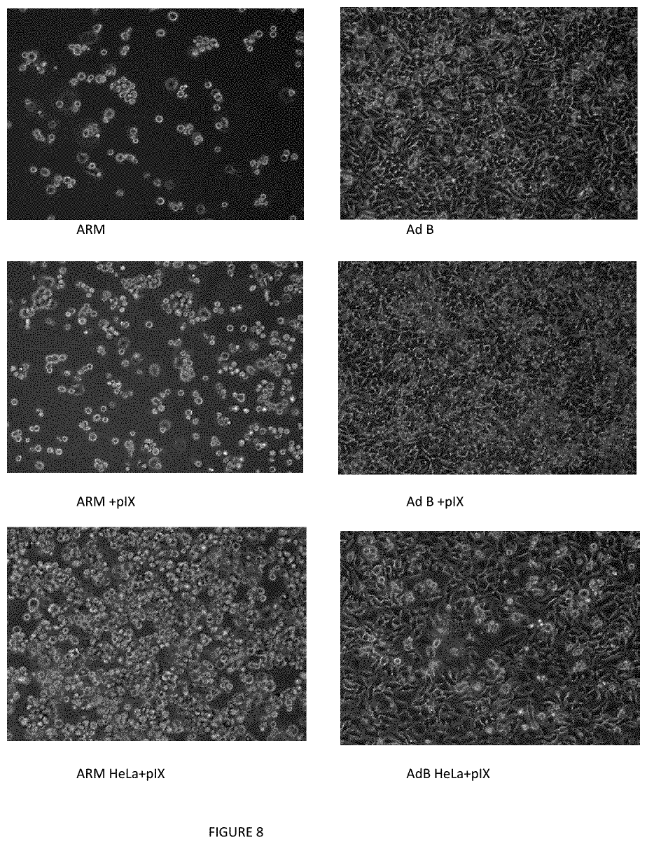

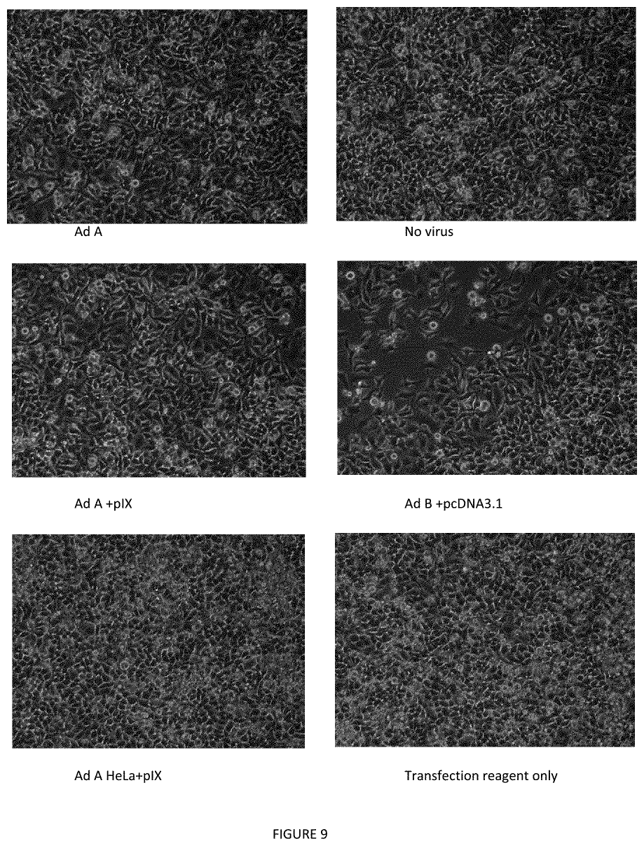

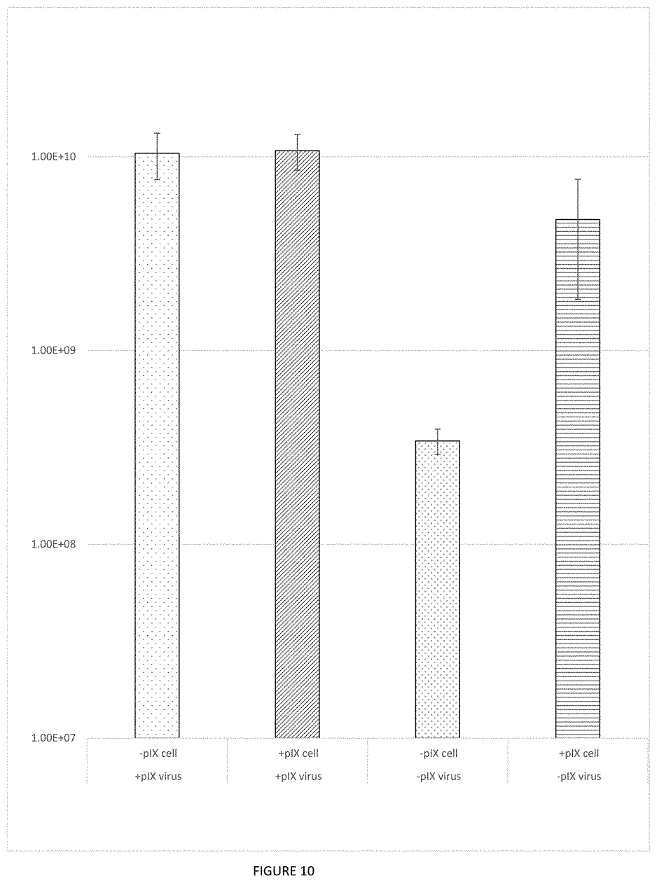

To study this, we used HeLa cells. The various types of adenovirus we used above cannot normally replicate in HeLa cells, because HeLa cells, unlike HEK293 cells, lack the necessary adenoviral complementation sequences. Wild-type adenovirus can, however, replicate in HeLa cells because wt virus is an RCA, and as such needs no complementation. We thus infected cells with comparable number of vectors or wild-type virus, and photographed the cells five days after infection. Our photographs show that the only cells showing signs of virus replication (visually appearing as rounded, floating cells) are the ones that were infected with wild-type adenovirus. In contrast, pIX itself did not lead to virus replication regardless of whether the pIX was coded for by the virus genome or expressed in a recombinant HeLa cell. We show these results in FIGS. 8 and 9. In addition, when media from the various wells was tested in a conventional infectivity assay, RCA was found only when we used adenovirus reference material (“ARM”, a wild type adenovirus) (data not shown).

### Example 9—pIX Increases Suspension Culture Yield

Our results above show that if produced in an environment that contains pIX, adenovirus are more infective and show improved infection kinetics, i.e., faster transduction of target cells, a given level of transduction achieved by fewer infective particles or plaque forming units, and a faster production of progeny adenovirus. Protein IX thus makes an improved adenoviral vector.

Protein IX expressed in producer cells also has another surprising benefit. The art teaches two general types of producer cell culture: adherent culture and suspension culture. The two share the common aim of providing cell cultures in which one can manufacture viruses. The two cell culture types, however, have two differences relevant here.

First, suspension cell culture is markedly less expensive than, and thus is preferable to, adherent culture.

Second, the two culture methods provide unpredictably-different yields: for certain adenovirus variants, adherent culture is far more efficient than suspension culture. See Example 2 above. Figuring out which cell culture approach most efficiently produces a particular adenovirus variant has to date been a matter of trial-and-error because the art does not identify any results-critical parameter(s) to predict which cell culture approach would be best to produce a given adenovirus.

We inadvertently, and surprisingly, discovered that the results-critical parameter. We tested the effect of stable pIX expression in producer cells in suspension culture. As discussed above, we found that transient transfection with a pX coding plasmid under control of the CMV promoter resulted in a high level of pIX expression. We then used these pIX-expressing cells to make an adenovirus which has an expressed pIX gene in its genome. We found that high levels of pIX in the producer cells increased the ratio of transduction units per virus genome (the “TU:vg” ratio) for the resulting vector. This implies that the pIX expressed by the producer cell improved the likelihood that the produced viral genomes would be packaged successfully. The overall effect on vector productivity, however, was not positive.

We reasoned that transfection stresses the producer cells. Also, the low number of infective units in the virus production in Example 7, after the “empty” pcDNA3.1 plasmid transfection, hints that the virus productivity suffers due to the transfection (this is not the only example we've seen of this phenomenon). We hypothesized that different pIX concentrations may show different outcomes. We also knew to expect that the stably pIX expressing cell line has lower pIX expression than transiently transfected cells. We thus set off to test the effect of low pIX expression in suspension cultures. Stably pIX-expressing HEK293 cells (constructed as described above) and normal HEK293 cells were adapted to suspension culture. We then grew these suspension-adapted cells in Corning® 50 mL mini bioreactors. Two bioreactors of both cell lines (HEK293 and HEK293+pIX) were infected with either adenovirus which contained a functional, expressed pIX gene, or adenovirus with a pIX-deleted genome. Three days after we infected the suspension cells with the adenovirus, we sampled the media and lysed the cells release any virus inside them. We measured virus genome titers from the media (this provides a measure of extracellular virus genomes), and also from crude harvest materials (this provides a measure of intracellular virus genomes). We then calculated the total productivity as extracellular+intracellular virus.

Materials and Methods for the Suspension Cultures

Adherently growing cells were adapted to suspension culture by detaching the adherent cells and using centrifugation (209-400×g, 5 min) to pellet the cells. The supernatant (adherent cell culture media) was removed and cells were suspended into suspension culture media (EX-CELL@ 293 Serum-Free Medium from Sigma-Aldrich). Cells were centrifuged again and the supernatant was removed. Cells were re-suspended into the suspension culture media and counted. After counting, cells were diluted into 5e5-1e6 cells/ml in 3-20 ml volumes and placed in 50 ml Mini Bioreactors, which were then grown on shaker platform (180 rpm shaking, tubes on 45-degree angle) inside a normal cell culture incubator. Cells were counted and/or observed 2-3 times per week and cultures were diluted with new media or the media was refreshed as described above. For the infections cells were seeded into 5×10⁵ cells/ml in 5 ml volumes. Cells were infected on the day following the seeding using 50 vg/cell and the infections were incubated for 3 days. 4 ml of cell suspension was taken into a test tube and cells were centrifuged 209×g, 5 min at 20° C. Supernatant was removed and sampled for ddPCR. Cell pellet was suspended in 3 ml PBS and stored at −80° C. ddPCR was performed after 3 freeze-thaw cycles as described earlier.

We found that virus which includes an expressed pIX gene is produced in about the same yield regardless of whether the suspension-culture producer cell expresses pIX; including a pIX plasmid to the producer cell increases yield only by 3%. In contrast, we found that virus which does not include an expressed pIX gene is produced in greatly different quantities depending on whether the suspension-culture producer cell expresses pIX; including a pIX plasmid to the producer cell increases yield by about 1,400%:

Protein IX Increases Suspension-Culture Viral Yield By About 1,400%

| | | Yield | Yield | | | |
|---|---|---|---|---|---|---|
| | Producer | vg/ml | vp/cell | | | Percent |
| Virus | Cell | (Mean) | split* | SD | SD % | Change |
| + | − | 1.04E+10 | 3231 | 2.81E+09 | 26.9 | |
| + | + | 1.08E+10 | 37121 | 2.24E+09 | 20.8 | 3% |
| − | − | 3.42E+08 | 80740 | 5.12E+07 | 15.0 | |
| − | + | 4.74E+09 | 83530 | 2.90E+09 | 61.2 | 1,388% |

+ = Contains stably-expressed pIX gene
− = Does not contain expressed pIX gene
vg/mL = viral genomes per mL of culture
vp/cell = viral particles per producer cell. *5 × 10⁵ cells/ml were split on the previous day

This increase in yield is significant because it enables the artisan to, for the first time. produce pIX-deleted adenovirus in suspension cell culture at yields similar to those achieved using adherent cell culture.

Where pIX is not expressed during viral production, then the adenovirus must likely be manufactured using the more expensive and cumbersome adherent cell culture approach. In contrast, where pIX is expressed during viral production (e.g., as an expressed part of the viral genome, or as a plasmid-borne pIX transgene in the producer cell), then one can achieve similar viral yield using the more economical and simpler suspension cell culture.

### Summary

All adenoviral gene therapy vectors, like Ad vector A here, do not contain pIX. In contrast, we surprisingly found that adenoviral gene therapy vector which includes higher than normal amount of pIX more rapidly infects, transduces and replicates in target cells. Our invention thus pertains to increasing the infectivity of adenoviral gene therapy vector by including super-physiological amounts of pIX on the vector.

For the avoidance of doubt, in our appended legal claims we use the term “expressible gene” to encompass a nucleic acid sequence which directly or indirectly produces a functional product. That functional product may be a polypeptide. Alternatively, the functional product may be an antisense RNA sequence, an siRNA sequence, or another type of functional RNA. Our use is consistent with that in the art. For example Wikipedia says, “a gene is a sequence of nucleotides in DNA or RNA that codes for a molecule that has a function. During gene expression, the DNA is first copied into RNA. The RNA can be directly functional or be the intermediate template for a protein that performs a function.” Similarly, NIH's website says, “Some genes act as instructions to make molecules called proteins. However, many genes do not code for proteins.” See https://ghr.nlm.nih.gov/primer/basics/gene.

Given out specific experimental results, the artisan can readily make equivalent variants or modifications. For example, while our specific experiments make adenovirus in adherent human producer cells, one can use a suspension line or insect cells to make equivalent adenovirus. Similarly, while our specific experiments here used wild-type pIX, the artisan can readily identify pIX analogs, variants and mutants which perform the same function in the same way to achieve the same result as wild-type protein here does. For example, a his-tagged version of protein IX has already been constructed in our laboratory. Similarly, for transgene the art teaches that short-form VEGF-D3, endostatin, angiostatin, thymidine kinase, human interferon alpha-2b, ABCA4, ABCD-1, myosin VIIA, cyclooxygenase-2, PGF2-alpha receptor, dopamine, human hemoglobin subunit beta and antibody subunits are suitable for use as transgenes in an adenovirus vector. We thus intend our patent's legal coverage to be defined by our legal claims and equivalents thereof, rather than by our specific examples.

1. An adenoviral gene therapy vector comprising adenovirus protein IX and an expressible transgene, said adenoviral gene therapy vector produced in a human cell which expresses adenovirus protein IX even when not infected or transduced by an adenovirus.

2. The adenoviral gene therapy vector of claim 1, wherein the adenoviral gene therapy vector comprises about twelve (12) adenovirus protein IX molecules per adenoviral gene therapy vector particle.

3. The adenoviral gene therapy vector of claim 1, wherein the human cell expresses adenovirus protein IX in a greater than stoichiometric amount.

4. The adenoviral gene therapy vector of claim 1, wherein the human cell produces a greater amount of adenovirus protein IX than does a similar human cell that has been infected with wild-type adenovirus at a Multiplicity of Infection of 1.

5. The adenoviral gene therapy vector of claim 1, wherein the adenoviral gene therapy vector is conditionally replicative.

6. The adenoviral gene therapy vector of claim 1, wherein the expressible transgene is, in a human patient, therapeutic.

7. The adenoviral gene therapy vector of claim 1, wherein the adenoviral gene therapy vector has a genome comprising a nucleic acid sequence that is idiosyncratic to adenovirus.

8. The adenoviral gene therapy vector of claim 1, wherein the adenoviral gene therapy vector has a genome which does not contain an expressed pix gene.

9. The adenoviral gene therapy vector of claim 1, wherein the adenoviral gene therapy vector has a genome comprising an adenoviral packaging signal which does not have a cre/lox site flanking it.

10. The adenoviral gene therapy vector of claim 1, wherein the vector is at least twice as infective, when measured 48 hours post-transformation, as the same vector produced in a similar producer cell which does not express adenovirus protein IX.

11. The adenoviral gene therapy vector of claim 1, wherein the vector shows a cytopathic effect on target cells at least about 25% faster than does the same adenoviral gene therapy vector produced in a similar producer cell which does not express adenovirus protein IX.

12. A mixture of the adenoviral gene therapy vector of claim 1 and non-infective adenoviral virus-like particles (VLPs), wherein the ratio of gene therapy vector to VLPs is greater than 1:100, where infectivity is measured by a plaque-forming assay.

13. The adenoviral gene therapy vector of claim 1, where the vector has a genome larger than 35 kb.

14. The adenoviral gene therapy vector of claim 1, wherein the human cell is grown in a non-adherent, suspension culture when producing the adenoviral gene therapy vector.

15. The adenoviral gene therapy vector of claim 1, where the producer cell comprises a plasmid having an expressed pix gene.

16. An adenoviral gene therapy vector having a genome comprising an adenoviral packaging signal which does not have a cre/lox site flanking it, produced in a producer cell which expresses adenovirus protein IX even when not infected or transduced by an adenovirus.

17. The adenoviral gene therapy vector of claim 16, wherein the adenoviral gene therapy vector comprises about twelve (12) adenovirus protein IX molecules per adenoviral gene therapy vector particle.

18. The adenoviral gene therapy vector of claim 16, wherein the human cell expresses adenovirus protein IX in a greater than stoichiometric amount.

19. The adenoviral gene therapy vector of claim 16, wherein the human cell produces a greater amount of adenovirus protein IX than does a similar human cell that has been infected with wild-type adenovirus at a Multiplicity of Infection of 1.

20. The adenoviral gene therapy vector of claim 16, wherein the adenoviral gene therapy vector is conditionally replicative.

21. The adenoviral gene therapy vector of claim 16, wherein the expressible transgene is, in a human patient, therapeutic.

22. The adenoviral gene therapy vector of claim 16, wherein the adenoviral gene therapy vector has a genome comprising a nucleic acid sequence that is idiosyncratic to adenovirus.

23. The adenoviral gene therapy vector of claim 16, wherein the adenoviral gene therapy vector has a genome which does not contain an expressed pix gene.

24. The adenoviral gene therapy vector of claim 16, wherein the adenoviral gene therapy vector has a genome comprising an adenoviral packaging signal which does not have a cre/lox site flanking it.

25. The adenoviral gene therapy vector of claim 16, wherein the vector is at least twice as infective, when measured 48 hours post-transformation, as the same vector produced in a similar producer cell which does not express adenovirus protein IX.

26. The adenoviral gene therapy vector of claim 16, wherein the vector shows a cytopathic effect on target cells at least about 25% faster than does the same adenoviral gene therapy vector produced in a similar producer cell which does not express adenovirus protein IX.

27. A mixture of the adenoviral gene therapy vector of claim 16 and non-infective adenoviral virus-like particles (VLPs), wherein the ratio of gene therapy vector to VLPs is greater than 1:100, where infectivity is measured by a plaque-forming assay.

28. The adenoviral gene therapy vector of claim 16, where the vector has a genome larger than 35 kb.

29. The adenoviral gene therapy vector of claim 16, wherein the human cell is grown in a non-adherent, suspension culture when producing the adenoviral gene therapy vector.

30. The adenoviral gene therapy vector of claim 16, where the producer cell comprises a plasmid having an expressed pix gene.

31-41. (canceled)

42. A mixture of the adenoviral gene therapy vector of claim 1 and non-infective adenoviral virus-like particles (VLPs), wherein the ratio of gene therapy vector to VLPs is greater than 1:100, where infectivity is measured by a plaque-forming assay.

43-45. (canceled)

46. A cell which expresses adenovirus protein IX even when not infected or transduced by an adenovirus, and which expresses adenovirus protein IX in a greater amount than does a similar cell that has been infected with wild-type adenovirus at a Multiplicity of Infection of not more than 1.

47. The cell of claim 46, where the cell is human.

48. The cell of claim 47, where the cell is a human embryonic kidney cell.

49. The cell of claim 46, wherein the cell further produces adenoviral gene therapy vector having a transgene.

50. The cell of claim 49, wherein the adenoviral gene therapy vector comprises about twelve (12) adenovirus protein IX molecules per adenoviral gene therapy vector particle.

51. The cell of claim 49, wherein the cell expresses adenovirus protein IX in a greater than stoichiometric amount.

52. The cell of claim 49, wherein the adenoviral gene therapy vector is conditionally replicative.

53. The cell of claim 49, wherein the expressible transgene is, in a human patient, therapeutic.

54. The cell of claim 49, wherein the adenoviral gene therapy vector has a genome comprising a nucleic acid sequence that is idiosyncratic to adenovirus.

55. The cell of claim 49, wherein the adenoviral gene therapy vector has a genome which does not contain an expressed pix gene.

56. The cell of claim 49, wherein the adenoviral gene therapy vector has a genome comprising an adenoviral packaging signal which does not have a cre/lox site flanking it.

57. The cell of claim 49, wherein the adenoviral gene therapy vector is at least twice as infective, when measured 48 hours post-transformation, as the same vector produced in a similar producer cell which does not express adenovirus protein IX.

58. The cell of claim 49, wherein the adenoviral gene therapy vector shows a cytopathic effect on target cells at least about 25% faster than does the same adenoviral gene therapy vector produced in a similar producer cell which does not express adenovirus protein X.

59. The cell of claim 49, wherein the cell further produces non-infective adenoviral virus-like particles (VLPs), and wherein the ratio of gene therapy vector to VLPs is greater than 1:100, where infectivity is measured by a plaque-forming assay.

60. The cell of claim 49, where the vector has a genome larger than 35 kb.

61. The cell of claim 46, grown in a non-adherent, suspension culture.

62. The cell of claim 49, grown in a non-adherent, suspension culture when producing the adenoviral gene therapy vector.

63. The cell of claim 46, wherein the cell comprises a plasmid having an expressed pix gene.

64. The cell of claim 49, wherein the cell produces more than about 3231 virus genomes per cell.

65. The cell of claim 49, wherein the cell produces at least about 4.7×10⁹ viral genomes per milliliter of culture media.

66. A suspension-cultured cell that expresses adenovirus protein IX even when not infected or transduced by an adenovirus.

67. The suspension-cultured cell of claim 66, wherein the cell expresses adenovirus protein IX in a greater amount than does a similar cell that has been infected with wild-type adenovirus at a Multiplicity of Infection of not more than 1.

68. The suspension-cultured cell of claim 66, where the cell is human.

69. The suspension-cultured cell of claim 69, where the cell is a human embryonic kidney cell.

70. The suspension-cultured cell of claim 66, wherein the suspension-cultured cell further produces adenoviral gene therapy vector having a transgene.

71. The suspension-cultured cell of claim 70, wherein the adenoviral gene therapy vector comprises about twelve (12) adenovirus protein IX molecules per adenoviral gene therapy vector particle.

72. The suspension-cultured cell of claim 70, wherein the cell expresses adenovirus protein IX in a greater than stoichiometric amount.

73. The suspension-cultured cell of claim 49, wherein the adenoviral gene therapy vector is conditionally replicative.

74. The suspension-cultured cell of claim 70, wherein the expressible transgene is, in a human patient, therapeutic.

75. The suspension-cultured cell of claim 70, wherein the adenoviral gene therapy vector has a genome comprising a nucleic acid sequence that is idiosyncratic to adenovirus.

76. The suspension-cultured cell of claim 70, wherein the adenoviral gene therapy vector has a genome which does not contain an expressed pix gene.

77. The suspension-cultured cell of claim 70, wherein the adenoviral gene therapy vector has a genome comprising an adenoviral packaging signal which does not have a cre/lox site flanking it.

78. The suspension-cultured cell of claim 70, wherein the adenoviral gene therapy vector is at least twice as infective, when measured 48 hours post-transformation, as the same vector produced in a similar producer cell which does not express adenovirus protein IX.

79. The suspension-cultured cell of claim 70, wherein the adenoviral gene therapy vector shows a cytopathic effect on target cells at least about 25% faster than does the same adenoviral gene therapy vector produced in a similar producer cell which does not express adenovirus protein IX.

80. The suspension-cultured cell of claim 70, wherein the suspension-cultured cell further produces non-infective adenoviral virus-like particles (VLPs), and wherein the ratio of gene therapy vector to VLPs is greater than 1:100, where infectivity is measured by a plaque-forming assay.

81. The suspension-cultured cell of claim 70, where the adenoviral gene therapy vector has a genome larger than 35 kb.

82. The suspension-cultured cell of claim 70, wherein the suspension-cultured cell comprises a plasmid having an expressed pix gene.

83. The cell of claim 70, wherein the cell produces at least about 4.7×10⁹ viral genomes per milliliter of culture media.

84. A method for manufacturing a pix-deleted adenoviral gene therapy vector in suspension cell culture, comprising: culturing in suspension cell culture a producer cell which expresses adenoviral protein IX even if not infected or transduced by adenovirus, transforming the cell with a pix-deleted adenoviral gene therapy vector genome, culturing the cell in suspension while the cell produces a pix-deleted adenoviral gene therapy vector, and then harvesting adenoviral gene therapy vector comprising adenovirus protein IX and a therapeutic transgene.

85. The method of claim 1, wherein the cell produces at least about 4.7×10⁹ viral genomes per milliliter.

86. An adenoviral gene therapy vector manufacturing process comprising obtaining human cells, transducing or transfecting those cells with expressible nucleic acid coding for adenovirus and with expressible nucleic acid coding for adenovirus protein IX, and with nucleic acid coding for a transgene, and then culturing the cells in suspension culture to produce adenoviral gene therapy vector comprising adenovirus protein IX and the transgene, and then harvesting the adenoviral gene therapy vector comprising adenovirus protein IX and the transgene.

87. A virus manufacturing process comprising obtaining human producer cells that express adenovirus protein IX, and then transducing or transfecting those cells with nucleic acid coding for an adenoviral gene therapy vector, and then culturing the cells in suspension culture to produce adenovirus protein IX and the adenoviral gene therapy vector, and then harvesting adenoviral gene therapy vector comprising adenovirus protein IX.

88. The manufacturing process of claim 87, wherein the nucleic acid coding for an adenoviral gene therapy vector is pix-negative

89. A manufacturing method for increasing the yield of adenoviral gene therapy vector, comprising manufacturing an adenoviral gene therapy vector in a producer cell that expresses adenovirus protein IX even when not infected or transduced by adenovirus and then harvesting adenoviral gene therapy vector, whereby the ratio of infective adenoviral gene therapy vector produced to viral genomes produced is at least about 20% greater than the ratio obtained when producing the same adenoviral gene therapy vector in a producer cell that does not expresses adenoviral Protein IX when not infected or transduced by adenovirus.

90. A human therapeutic method comprising administering to a human the adenoviral gene therapy vector of claim 1.


SEQUENCE LISTING (8 sequences)

SEQ ID NO: 1 - DNA, 26 bp, Adenovirus type 5
gccatgagcaccaactcgtttgatgg

SEQ ID NO: 2 - DNA, 18 bp, Adenovirus type 5
tagaaggcacagtcgagg

SEQ ID NO: 3 - DNA, 26 bp, Adenovirus type 5
gccatgagcaccaactcgtttgatgg

SEQ ID NO: 4 - DNA, 18 bp, Adenovirus type 5
tagaaggcacagtcgagg

SEQ ID NO: 5 - DNA, 20 bp, Adenovirus type 5
taatacgactcactataggg

SEQ ID NO: 6 - DNA, 22 bp, Adenovirus type 5
catgaccttatgggactttcct

SEQ ID NO: 7 - DNA, 21 bp, Adenovirus type 5
ctatccacgcccattgatgta

SEQ ID NO: 8 - DNA, 15 bp, Adenovirus type 5
ccatggtgatgcggt